(19) United States
(12) Patent Application Publication
(10) Pub. No.: US 2026/0048176 A1
(43) Pub. Date: Feb. 19, 2026

(54) MUSCLE ASSISTANCE MEMBRANE

(71) Applicants: UNIVERSITE DE LORRAINE, NANCY (FR); CENTRE NATIONAL DE LA RECHERCHE SCIENTIFIQUE, PARIS (FR)

(72) Inventors: Jean-Philippe JEHL, HOUDEMONT (FR); Gérôme GAUCHARD, VARANGEVILLE (FR); Franck CLEYMAND, FLAVIGNY-SUR-MOSELLE (FR); Mélanie Maria LEROUX, VANDOEUVRE-LES-NANCY (FR); Aurelia POERIO, NANCY (FR)

(21) Appl. No.: 18/996,287

(22) PCT Filed: Jul. 18, 2023
(86) PCT No.: PCT/EP2023/069892

§ 371 Date: Jan. 17, 2025

(30) Foreign Application Priority Data
Jul. 26, 2022 (FR) 2207683

(51) Int. Cl. A61L 27/20; A61F 2/08; A61H 1/02; A61L 27/36

(52) U.S. Cl. CPC A61L 27/20; A61F 2/08; A61H 1/02; A61L 27/3637; A61F 2002/0894; A61H 2201/169; A61L 2430/20; A61L 2430/30

(57) ABSTRACT

The invention relates to a muscle membrane intended to be affixed to a muscle of a human or animal body, the membrane being capable of at least partially surrounding the muscle, the muscle comprising fibres covered with an outer tissue, the positioned membrane having a shape that is identical to that of the muscle. The membrane is characterised in that it exhibits anisotropic behaviour so as to reproduce the mechanical behaviour of the muscle, the membrane comprising: -a matrix designed in a biocompatible material having an elasticity similar to that of the outer tissue of the muscle, -one or more reinforcing strips rigidly attached to the matrix, each strip being designed in a biocompatible material having an elasticity similar to that of the fibres it covers and each strip being oriented according to membrane stiffening requirements and according to the expected muscle correction.

### TECHNICAL FIELD OF THE INVENTION

The invention relates to a membrane configured to be affixed to a muscle of a human or animal body, the membrane being capable of at least partially surrounding the muscle.

The invention also concerns a method for manufacturing such a membrane by 3D printing.

The invention also concerns a prosthesis for a muscle, such as a cardiac prosthesis or muscular prosthesis, formed by such a membrane.

The invention also concerns an orthosis, such as a plantar orthosis or forearm orthosis, formed by such a membrane.

Finally, the invention concerns the use of such a membrane on a muscle of a human or animal body.

The technical field of this innovation also concerns the health sector, and more specifically bio-prosthesis and bio-orthosis.

### TECHNICAL BACKGROUND

Whether we are talking about the heart or a skeletal muscle, when a muscle is atrophied or damaged, the movement for which it is intended will be prevented or compensated for by biological adaptation (e.g. fibrosis, extension of the muscle) or physiological adaptation (e.g. compensation of the movement by the body or by the neighbouring muscles).

### Case of the Heart Muscle

The myocardium infarction is caused mainly by a blockage in the coronary arteries that supply oxygen to the heart muscle, resulting in a lack of oxygen (ischaemia) in certain areas of the heart muscle. The ischaemia in the heart muscle does not irrevocably cause an infarction, but may lead to mild heart failure or cardiogenic shock. The result is an alteration in the contraction and relaxation capacity of the heart muscle (hypokinesia or akinesia) in certain regions of the heart.

A myocardium infarction will be diagnosed when the assessment indicates that there is necrosis of certain regions of the heart muscle as a result of an ischaemia. The infarction most often affects the left ventricle, but the right ventricle may also be affected.

The infarction of the myocardium leads to a biological and physiological changes in the heart (remodelling). This leads to changes in the size, thickness and shape of the left ventricle. The risk of long-term mortality is associated with the extent of this remodelling. The infarction may also be a source of mechanical complications, leading to a heart failure and cardiac rhythm disorders, which carry the risk of sudden death. Even with treatment, the occurrence of an infarction of the myocardium increases the risk of mortality in 15% of patients. The patient care often involves heavy treatment, regular medical monitoring and lifestyle changes, the surgery or the fitting of an implantable cardioverter defibrillator or a pacemaker.

The biomedical materials configured to an epicardial application have been studied intensively in recent years for a variety of therapeutic purposes.

Most biomaterials configured to the epicardial applications are manufactured in the form of membranes or patches.

For example, thin membranes containing various biological properties are applied directly to the target epicardial region.

They are also haemostatic patches, creating an interface to prevent a severe tissue adhesion after a cardiac surgery.

Or cardiac patches delivering a cell therapy after an infarction of the myocardium.

However, the research in this field has mainly focused on the biological or therapeutic behaviour of these biomaterials, largely ignoring analysis of their mechanical interaction with the heart.

The shape and the isotropic behaviour of the membranes and patches are not well suited to the movement of the myocardial wall. The movement of the myocardial wall during systole and diastole produces a complex force in different directions.

Once the biomedical material adheres to the epicardium, it undergoes the same cyclic deformation and the same stress caused by the heartbeat as the epicardium. The structure of the muscle fibres also differs from one area to another, exerting a mechanical force in different directions during the cardiac cycle.

Most epicardial biomaterials are manufactured as isotropic membranes or patches that may not adapt to the specific structure of the heart muscle fibres, thus potentially applying an inappropriate force on the epicardium, which could eventually cause a restriction effect.

The current membranes and patches may therefore have a mechanical influence on the heart, which is not necessarily desirable as it goes against the natural movement of the organ.

### Case of the Skeletal Muscle

In the case of the skeletal muscles, whose function is mainly to move the limbs, several types of injury may be observed (stretching, straining, tearing, atrophy, etc.). In the case of disabled people, a serious injury to the nervous system and/or muscle, for example, leads to a drastic reduction in movement of the limb, to the point of immobility, with this immobility eventually leading to a muscle wasting and a muscle atrophy. The loss or the reduction in mobility of a limb may not be compensated for, or only with difficulty.

Orthoses are available to correct the movement when the muscle is no longer able to do so. The construction of the orthosis may be adapted to the individual's needs (for example: more rigidity, less torsion, more traction, etc.) to compensate for reduced mobility, facilitate movement and/or reduce the pain associated with the disability.

However, the current orthoses are designed in a single material, with isotropic behaviour. The orthosis does not adapt to the different regions it surrounds, to the different fibre orientations that differ from one point of the organ to another. A textile orthosis made of synthetic fibre stiffens the movement only in the direction of extension of the tissue, which does not necessarily correspond to the directions of all the muscle fibres surrounded by the orthosis.

The objective of the present invention is to design a muscle membrane from compatible materials of natural or synthetic origin, with the aim of reducing the compensation and/or repairing the damaged muscles, whether the heart muscle or the skeletal muscle. This membrane must be capable of following the complex movement of the muscle without mechanically influencing the desired movement and correcting the deleterious movements. It must therefore be able to deform to match the expected deformation of the muscle in action.

### SUMMARY OF THE INVENTION

This aim is achieved by means of a muscle membrane configured to be affixed to a muscle of a human or animal body, said membrane being capable of at least partially surrounding the muscle, the muscle comprising fibres covered with an external tissue, the positioned membrane having a shape that is identical to that of the muscle.

This membrane is characterised in that it exhibits an anisotropic behaviour so as to reproduce the mechanical behaviour of the muscle, the membrane comprising:
a matrix designed in a biocompatible material having an elasticity similar to that of the external tissue of the muscle,
one or more reinforcing strips associated with the matrix, each strip being designed in a biocompatible material having an elasticity similar to that of the fibres it covers, and each strip being oriented according to the stiffening requirements of the membrane.

The main idea behind this invention is to design a biomaterial membrane that takes into account not only the shape but also the mechanical properties of the muscle, i.e. the external muscle tissue and muscle fibres.

In fact, this membrane is designed to be placed around a muscle, surrounding it in a very precise way, with an exact shape.

This membrane is designed to deform with the muscle by exerting an appropriate mechanical force on it. It forms a kind of double skin, which follows and corrects the natural movement of the muscle.

This membrane is affixed directly to the external tissue of the muscle, and must therefore mainly follow and correct the movement of this external tissue. It is a cellular tissue. In the case of skeletal muscle, this external tissue is the skin. In the case of heart muscle, this external tissue is the epicardium.

More specifically, the matrix of the membrane is in direct contact with the external tissue. The matrix must therefore reproduce the mechanical properties of this external tissue.

Mechanical properties refer to the elasticity of the tissues and of the fibres. The first biocompatible material forming the matrix has an elasticity corresponding to that of the external tissue.

The matrix is designed in a first biocompatible material with a first elasticity similar to the elasticity of the external tissue, giving the membrane an initial isotropic mechanical behaviour.

This may be modified by combining reinforcement strips of different elasticity or of identical elasticity but locally increasing the shape of the membrane. These reinforcements modify the isotropic behaviour of the membrane into an anisotropic and more particularly transverse isotropic or orthotropic behaviour.

It is advantageous not just to focus on the external tissue, but also to take into account the orientations of the fibres located just behind the external tissue, as they make a major contribution to the movement of the organ in action.

The elasticity of the muscle varies from one area to another depending on the fibre orientation.

The fibres with the same orientation will have the same elasticity.

It is therefore essential to analyse the orientation and the elasticity of the fibres locally, in order to be able to precisely design the mechanical properties of the membrane area by area.

After a precise analysis of the muscle fibres, several types of areas are defined:
areas that need to be reinforced, or encircled, depending on the pathology or the care we wish to provide via the membrane.
areas where the direction of extension of the fibres is similar to the main direction of extension of the external tissue: this means in particular that the elasticities are similar.
areas where the direction of extension of the fibres is different from the main direction of extension of the external tissue: this is called the secondary direction of extension. This means that the elasticities are different.

The membrane must adapt to all these areas thanks to the reinforcement strips associated with the matrix, which serve to reinforce and orientate the membrane as best it may, adapting as closely as possible to the muscle.

The orientation of the membrane is performed depending on the stiffening requirements of the membrane, which in turn depend on the muscular correction we wish to make to the muscle.

In one possible configuration, the matrix is created in a first step and the reinforcing strips are added to the matrix in a second step. They are then attached to the matrix by attachment means. In this case, two layers are superimposed: the layer of the matrix and the layer of the strip.

The attachment means may be of the overmoulding, clip or adhesive type, etc.

When the strips are superimposed on the matrix, the result is two superimposed layers forming a reinforcement area, i.e. an area where the matrix is stiffened.

These reinforcement strips allow the thickness of the membrane to be varied. The membrane will be thin in areas that you don't want to impact, and thicker in areas that you do want to impact, for example in areas of muscle weakness. In another possible configuration, the matrix and the reinforcing strips are created at the same time, during the manufacture of the membrane. The material of the strips may be integrated into the material of the matrix during manufacture to “fill in the gaps” and provide a different mechanical characteristic in a more localised way in the membrane.

As previously mentioned, each strip is made from a material with a specific elasticity. In this way, several strips of different materials are combined with the matrix so as to obtain a membrane that best corrects the mechanical behaviour of the muscle or of the organ.

According to the invention, some strips are made of a material identical to the material of the matrix, and some strips are made of another material. It all depends on how elastic you want the strip to be.

The anisotropic behaviour of the membrane may be achieved with the same material for the matrix and the reinforcing strips, or with different materials for the matrix and the strips. Generally speaking, the presence of reinforcing strips modifies the mechanical behaviour of the membrane, making it anisotropic, i.e. with properties that vary according to the direction and the areas.

The biocompatible materials may be of natural or synthetic origin.

They are chosen from latex, synthetic fibres, natural gums such as chitosan, tamarind, alginate, gelatin, guar, xanthan, or mixtures of these gums, and a polymer of the polyacrylamide type.

The natural gums are non-toxic and have different viscosity properties depending on their type, giving them a different rigidity after drying.

These materials are chosen taking into account the specific structure of the muscle tissue and the orientation of the fibres.

Preferably, the membrane is designed by a mixture of chitosan and guar, or a mixture of chitosan and tamarind.

Preferably, each reinforcement strip may be adapted to the requirements, by choosing the material, the tension and the orientation.

Each strip therefore has a specific tension within the membrane.

Voltage refers to the voltage applied between the two ends of the strip, attached or integrated into the matrix.

By orientation we mean the orientation of the strip on the matrix in correlation with its positioning on the muscle.

The materials of the strips are chosen according to the desired anisotropy of the membrane, the strips are oriented according to the orientation of the fibres they cover, and the strip tension is applied according to the desired mechanical behaviour of this area. The combination of these three parameters results in a membrane that corrects the movement of the muscle it surrounds.

Advantageously, the membrane is obtained by moulding.

Advantageously, the membrane is obtained by weaving one or more textile fibres.

Advantageously, the membrane is obtained by 3D printing. The muscle membrane is then produced using cutting-edge technology.

Advantageously, the membrane has an internal surface suitable for coming into contact with the muscle, and said internal surface may be functionalised. This functionalisation is carried out using controlled delivery systems that may or may not contain active ingredients and/or biomolecules.

For example, such a biological functionalisation may be achieved by integrating cells, growth factors, nutrients and/or therapeutic agents.

The invention also relates to a method for manufacturing a membrane configured to a use on a muscle of a human or animal body, comprising the following steps:
scanning the muscle to be surrounded to define its outer shape and the orientation of its fibres;
modelling the shape, elasticity and directions of extension of the membrane which is to surround the muscle at least partially;
forming a support taking on the shape of the muscle;
forming the membrane from at least one biocompatible material to form the matrix and the reinforcing strips.

Thanks to this scanning technique, the membrane may be customised to fit the exact shape of the muscle of the patient.

The membrane may be formed by any conventional technique, for example by moulding, or by 3D printing with injection of the biocompatible material or materials.

In the case of moulding, a support in the form of a mould is first formed, taking the exact shape of the muscle. Then, in a second step, the biocompatible material is poured into the mould, and the membrane is formed, following the shape of the mould.

In 3D printing, a support in the exact shape of the muscle is printed at the same time as the membrane, layer by layer, with the membrane 1 covering the support. The material used for the support is not the same as that used for the membrane.

The steps of forming the support (2) and the membrane (1) are carried out by 3D printing, the membrane (1) being printed simultaneously with the support (2).

The invention relates to a prosthesis for a muscle, of the cardiac prosthesis or muscular prosthesis type, formed by a membrane as described above.

In this case, the membrane is deployed, for example, using a minimally invasive operation.

The matrix of the membrane will have an elastic behaviour as close as possible to the extracellular tissue of the heart for the cardiac prosthesis.

Such a muscle-assist cardiac prosthesis reproduces the shape and the orientation of the fibres of the heart with specific mechanical functions (elasticity). It may be used to reduce deformation of the ventricle (particularly magnification) following infarction, and/or to act as an interface for a mechanical exoskeleton or to repair damaged areas by functionalising the membrane.

The invention also relates to an orthosis for a muscle, of the plantar orthosis or forearm orthosis, formed by a membrane as described above.

The matrix of the membrane will have an elastic behaviour as close as possible to the skin for the orthosis. Thanks to its mechanical properties, the membrane corrects the movement when the muscle is no longer able to do SO.

Finally, the invention relates to a use of a membrane, as described above, on a muscle, said membrane at least partially surrounding the muscle.

### BRIEF DESCRIPTION OF THE FIGURES

Further characteristics and advantages of the invention will become apparent from the following detailed description, for the understanding of which reference is made to the attached drawings wherein:

FIG. 1 is an example of a forearm orthosis.

FIG. 2 is another example of a forearm orthosis.

FIG. 3 shows the orthosis shown in FIGS. 1 and 2 in place on a forearm.

FIG. 4 is a perspective view of a mould of an organ (in this case the heart) on which a membrane is printed;

FIG. 5 is a perspective view of a membrane (in this case a cardiac membrane) configured to be positioned on an organ (in this case the heart);

FIGS. 6A, 6B and 6C illustrate the fibrous directions of an organ (in this case a heart);

FIGS. 7A, 7B, 7C, 7D and 7E show various steps in the creation of a cardiac membrane.

### DETAILED DESCRIPTION OF THE INVENTION

In the following description, elements with an identical structure or similar functions will be designated by same references.

The invention concerns a membrane of biocompatible material, suitable for at least partially surrounding a muscle of a human or animal body, in order to provide temporary and/or permanent functional correction.

In this detailed description, several examples of membranes will be presented.

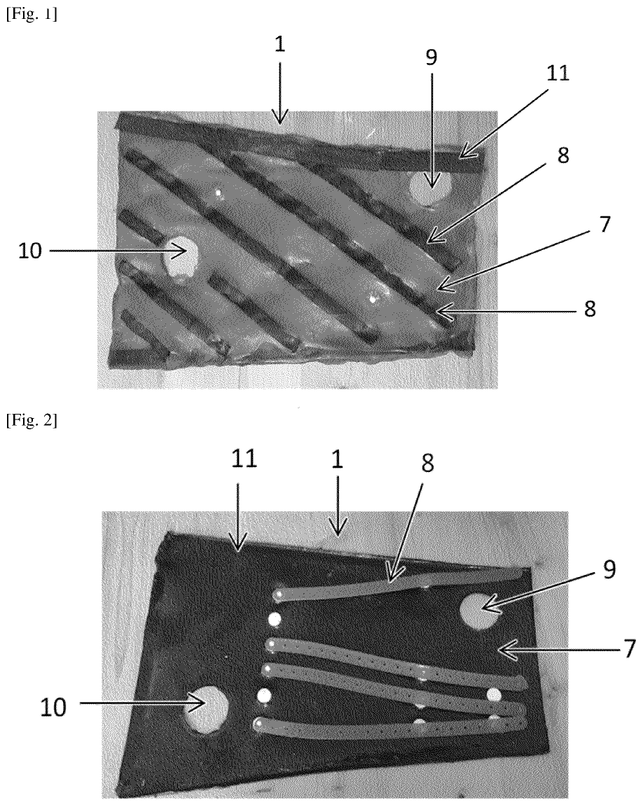

FIGS. 1 to 3 show examples of a forearm membrane used as orthosis.

In FIG. 1, the orthosis comprises a matrix 7 made of Latex and reinforcement strips 8 made of synthetic tissue integrated into the matrix 7 during manufacture. Other materials fall within the scope of the present invention.

In FIG. 2, the orthosis comprises a matrix 7 made of Latex and Latex reinforcement strips 8 superimposed after to the matrix 7. These strips 8 are attached to the matrix 7, in this case using a clip. Other materials and attachment means are covered by the present invention.

During the attachment, the orientation of the strips 8 is chosen so as to correspond to the desired correction as a function of the orientation of the muscle fibres located underneath when the orthosis is put in place.

During the attachment, the tension applied between the two ends of the strip 8 may be adjusted. To do this, the strips 8 are pre-drilled to form notches, and depending on the notch chosen for the clip, the strip 8 will be more or less tense.

The strips 8 are attached to the matrix 7 at different heights (or locations), giving different intensities and orientations to the force applied to the forearm. The orthosis thus manufactured in FIGS. 1 and 2 has anisotropic behaviour and corrects the movements of the damaged muscle (e.g. atrophied muscle). The reinforcement strips 8 induce a resistance on specific arm movements to allow the forearm to maintain a functional position and/or return to its initial position.

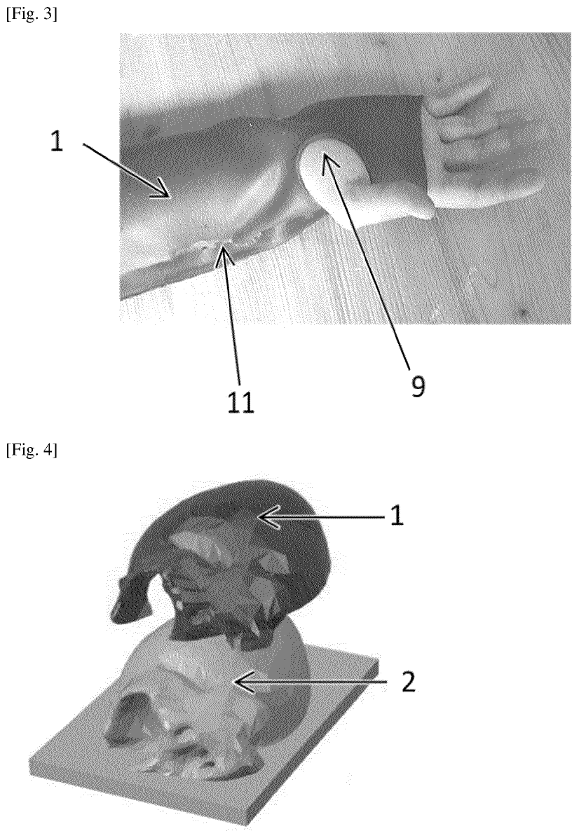

FIG. 3 shows the orthosis shown in FIGS. 1 and 2 in place on the forearm. This orthosis comprises an orifice 9 for the thumb to pass through, and an orifice 10 for the elbow to pass through.

Here, the membrane is manufactured flat, from a mould into which the latex is poured and then dried under controlled temperature. Velcro strips 11 are arranged on the longitudinal sides, so that they may be joined together once the orthosis has been positioned around the forearm. The present invention also comprises other attachment means, in particular a permanent assembly to form a sleeve-type orthosis.

FIGS. 4 to 6 show an example of a membrane used as a cardiac prosthesis. The heart has a limited capacity for regeneration. Once the cardiomyocytes (the contractile cells of the heart muscle) in an adult heart are damaged, they are replaced by non-contractile fibrous scar tissue. The loss of the contractile capacity of the cardiomyocytes leads to a heart dysfunction and ultimately a heart failure. A promising approach to the treatment of the infarction of the myocardium is the application of two-dimensional (2D) or three-dimensional (3D) membranes.

The three-dimensional membrane according to the invention is produced from a scan of the heart of a patient in order to obtain a suitable shape.

This scan gives the exact dimensions of the heart. From this scan, we may define the exact dimensions of the membrane that will cover this heart.

The membrane must have a shape that corresponds to the external shape of the heart, to fit it snugly.

In order to manufacture cardiac membranes with the appropriate mechanical characteristics, it is necessary to study the cardiac tissue.

A heart is shown in FIG. 6C.

Its heart tissue is shown in FIG. 6A and breaks down into:
i) an epicardium 4 (corresponding to an outer envelope)
ii) a myocardium 5 (corresponding to muscle fibres, referred to as myofibres)
iii) an endocardium 6 (which corresponds to an inner envelope).

The myocardium 5 is rolled up and roughly forms an 8.

More specifically, there is a helical orientation of the myocardium 5, from the base (i.e. the upper part in FIG. 6C) to the apex of the heart (i.e. the lower part in FIG. 6C).

When the heart is scanned in FIG. 6C, the different directions in which the fibres extend may be seen. When the heart muscle is solicited, it will stretch or contract depending on the direction of the fibres.

In the case of the heart, as illustrated in FIG. 6B, there are myofibres which extend along:
a first extension direction, referred to as the main extension direction, radially, symbolised by the arrow R;
a second extension direction, referred to as the secondary extension direction, longitudinally, symbolised by the arrow L.

It may be seen here that the major part of the myocardium 5 has a radial fibrous orientation.

The zoom in FIG. 6B illustrates the orientation of the myofibres over a longitudinally extending area of the myocardium 5.

Moving upwards from the apex to the base, the fibres of the myocardium 5 may be seen to be oriented alternately longitudinally and radially.

There are variations in the elasticity of the cardiac tissue depending on the elements considered (epicardium 4, myocardium 5 and endocardium 6), and also depending on the fibrous orientations of the myocardium 5.

These elasticity values are measured, for example by nanoindentation. The reduced Young's modulus is measured as a function of the penetration depth of the indenter on samples of the heart tissue.

This gives a certain elasticity value for a certain fibre orientation. In fact, one elasticity value is obtained for the myocardium 5 with a longitudinal orientation of the myofibres, and another elasticity value is obtained for the myocardium 5 with a radial orientation of the myofibres.

A certain value of elasticity is also obtained for the epicardium 4 and the endocardium 6.

To ensure that the cardiac membrane 1 reproduces the mechanical behaviour of the heart as closely as possible, it must be designed from one or more materials whose elasticity corresponds to that measured on the cardiac tissue and fibres.

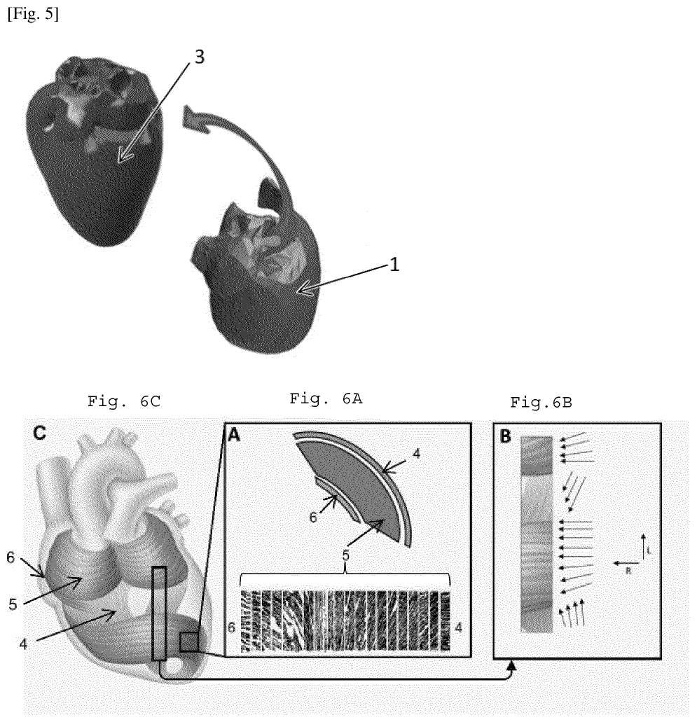

FIGS. 4 and 5 illustrate a cardiac membrane 1. These figures will be described in more detail below.

The cardiac membrane 1 mainly comprises a matrix 7 designed in a first material, the behaviour of which is similar to that of the epicardium 4 in FIG. 6.

More specifically, a first biocompatible material is chosen which has an elasticity similar to that of the epicardium 4. A first biomaterial with mechanical properties closest to those of the epicardium 4 is thus chosen to form a matrix 7, which will then have a first direction of extension.

For the reinforcement strips 8, the second biocompatible material is chosen, and they are positioned perpendicular to the covered fibres over all or part of the organ, depending on the movement correction requirements.

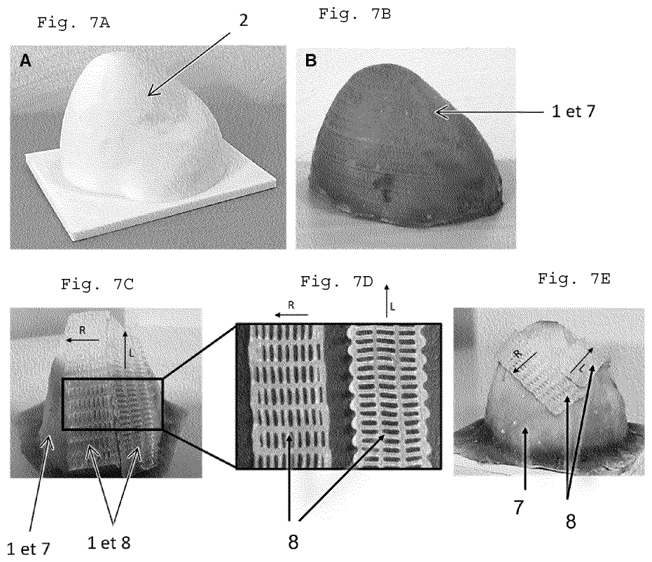

To create a heart membrane in the form of a sock, a basement 2, as shown in FIGS. 4 and 7A, is made from plastic by 3D printing following a scan of the heart, and then the membrane 1 is cast onto the basement 2 to take the shape of the heart, as shown in FIGS. 4 and 7B.

For example, the matrix 7 is made from alginate, chitosan and Guar gum, and the reinforcing strips 8 of the membrane 1 are designed in the same material in a concentration, ratio and/or oriented structure, more or less honeycombed as shown in FIGS. 7C, 7D and 7E, adapted to the stiffening required.

FIG. 7E shows in particular the positioning of such oriented honeycomb structures, which are arranged in precise directions according to the direction of extension of the fibres they cover, L or R.

It is also possible to design the membrane using 3D (three-dimensional) printing.

To give it this exact shape, it is necessary to print a basement and the membrane at the same time.

So, as shown in FIG. 4, following the heart scan, the basement 2 is printed stratum par stratum reproducing the external shape of the heart and the membrane 1. This basement 2 is printed with a plastic material that allows the basement to be rigid and act as a support for the printing of the membrane 1.

The cardiac membrane 1 is printed in one or more materials that allow it to be more or less flexible after printing. In the end, the latter perfectly matches the outer shape of the heart 3, as shown in FIG. 5.

Each stratum (layer) of the membrane 1 is printed using the two materials of the matrix 7 and the reinforcement 8, i.e. the two biocompatible materials.

The result is the cardiac membrane 1 as shown in FIG. 5.

The membrane 1 is reinforced by the organisation of the reinforcements 8. The orientation, nature and number of the reinforcements 8 are determined by analysis of the scan of the failed organ.

If only one area of the matrix 7 is to be reinforced, the reinforcing strips 8 are printed in the matrix 7.

The result is a membrane 1 with anisotropic behaviour composed of two interwoven materials, forming one or more localised reinforcements. These reinforcing strips 8 constitute means for stiffening the membrane 1.

If it is desired to change the orientation of the membrane 1 in different areas, then reinforcing strips 8 are locally printed on the matrix 7, using a biomaterial adapted to the desired orientation.

In the example shown, the matrix 7 has an elasticity similar to the longitudinal elasticity of the fibres of the myocardium. The same first biomaterial will therefore be used to print this matrix 7 and a second material in the form of reinforcement strips 8 whose elasticity corresponds to the radial elasticity of the fibres of the myocardium with an orientation perpendicular to the fibres, see FIG. 7.

If we print strips 8 in the form of a structure made up of cells of varying sizes FIG. 7, we obtain a cardiac membrane 1 with an anisotropic behaviour. The two materials used thus contribute to achieving the mechanical behaviour of the cardiac membrane 1 that is compatible with the heart to be treated.

The matrix 7 and the reinforcement strips 8 may be designed in polymer-based materials.

According to the invention, ink formulations for 3D printing based on natural gums have been developed for the manufacture of these anisotropic cardiac membranes 1.

Four ink formulations based on chitosan (CH), chitosan/guar gum (CH-GG), chitosan/tamarind gum (CH-TG) and alginate/polyacrylamine (AL-PO) were developed.

To improve the printability of these four inks, gelatin may be added to the composition of the inks.

The first ink is composed of 90/10 chitosan (90% deacetylation degree, 10 mPa·s average viscosity) at a concentration of 2% called CH.

The second ink is a mixture (ratio 1:1) of chitosan (2%) and Guar gum (2%) called CH-GG.

The third ink is a mixture (ratio 1:1) of chitosan (2%) and Tamarind gum (2%) called CH-TG.

The fourth ink is a mixture (ratio 8:1) of alginate (12.25%) and polyacrylamine (1.75%).

A printed membrane 1 based on CH-GG or CH-TG has an elasticity similar to that of the myocardium 5 with a radial orientation of the fibres.

A printed membrane 1 based on CH or AL-PO has an elasticity similar to that of the epicardium 4 and to that of the myocardium 5 with a longitudinal orientation of the fibres.

Preferably, the first biocompatible material is CH or AL-PO, and the second biocompatible material is CH-GG or CH-TG.

Preferably, all 3D constructs were printed using an Envisiontec bioprinter, with a 250 mm inner diameter needle and a constant speed of 5mm/s, while the pressure was adapted to each formulation: approximately 0.6 bar for CH, 0.8 bar for CH-TG and 1.0 bar for CH-GG. These settings may be transposed and adjusted to suit the printer used.

To be effective in restoring the cardiac function, the membranes 1 must: i) have mechanical properties similar to those of the heart, as seen above, and ii) optionally release therapeutic ingredients such as growth factors and/or biomolecules.

The functionalization of the membrane 1 falls within the scope of the present invention. In this case, the inner wall of the membrane 1 in contact with the heart is functionalised, at least in the areas to be treated.

Several applications are possible with such membranes 1.

These membranes 1 may be used as active cardiac prostheses (for example with the integration of cardiomyocytes) and passive cardiac prostheses made of polymers.

These membranes 1 may be used as muscular prostheses, or as orthoses, for example for top-level handisport athletes. It may be a forearm orthosis. The configurations shown in the cited figures are only possible examples, in no way limiting, of the invention which, on the contrary, encompasses the variations of shapes and designs within the reach of the person skilled in the art.

1. A muscle membrane configured to be affixed to a muscle of a human or animal body, said membrane being capable of at least partially surrounding the muscle, the muscle comprising fibres covered with an external tissue, the positioned membrane having a shape that is identical to that of the muscle, said membrane being characterized in that it exhibits an anisotropic behaviour so as to reproduce the mechanical behaviour of the muscle, the membrane comprising:
a matrix designed in a biocompatible material having an elasticity similar to that of the external tissue of the muscle,
one or more reinforcing strips associated with the matrix, each strip (8) being designed in a biocompatible material having an elasticity similar to that of the fibres it covers and each strip being oriented according to the stiffening requirements of the membrane

2. The membrane according to claim 1, characterised in that the biocompatible materials are chosen from latex, synthetic fibres, natural gums such as chitosan, tamarind, alginate, gelatine, guar, xanthan, or mixtures of these gums, a polymer of the polyacrylamide type.

3. The membrane according to claim 2, characterised in that it is designed by a mixture of chitosan and guar, or a mixture of chitosan and tamarind.

4. The membrane according to claim 1, characterised in that each strip has a specific tension within the membrane.

5. The membrane according to one of the claim 1, characterised in that it is obtained by 3D printing.

6. The membrane according to claim 1, characterised in that it has an internal surface suitable for coming into contact with the muscle, said internal surface being functionalised.

7. A method for manufacturing a membrane according to claim 1, comprising the following steps:
scanning the muscle to be surrounded to define its outer shape and the orientation of its fibres;
modelling the shape, elasticity and directions of extension of the membrane which is to surround the muscle at least partially;
forming a support taking on the shape of the muscle;
forming the membrane from at least one biocompatible material to form the matrix and the reinforcing strips

8. The method according to claim 7, characterised in that the steps of forming the support and the membrane are carried out by 3D printing, the membrane being printed simultaneously with the support.

9. A prosthesis, of the cardiac prosthesis or muscular prosthesis type, formed by a membrane according to claim 1.

10. An orthosis for a muscle, of the plantar orthosis or arm orthosis type, formed by a membrane according claim 1.

11. A use of a membrane according to claim 1 on a muscle, said membrane at least partially surrounding the muscle.
